(19) United States
(12) Patent Application Publication
(10) Pub. No.: US 2019/0315891 A1
(43) Pub. Date: Oct. 17, 2019

(54) THIOXANTHONE DERIVATIVE PHOTOINITIATOR

(71) Applicants: Henkel IP & Holding GmbH, Duesseldorf (DE); Henkel AG & Co. KGaA, Duesseldorf (DE)

(72) Inventors: Zhuming Shi, Shanghai (CN); Zhenhua Ju, Shanghai (CN); Zheng Lu, Shanghai (CN); Jiangbo Ouyang, Wallingford, CT (US); Jinqian Chen, Shanghai (CN)

(21) Appl. No.: 16/452,592

(22) Filed: Jun. 26, 2019

Related U.S. Application Data
(63) Continuation of application No. PCT/CN2016/112528, filed on Dec. 28, 2016.

(51) Int. Cl. C08F 2/48; C07D 335/16; C09J 5/06

(52) U.S. Cl. CPC C08F 2/48; C09J 2479/08; C09J 5/06; C07D 335/16

(57) ABSTRACT

This invention relates to a thioxanthone derivative photoinitiator, a radiation curable composition comprising the same and the use thereof. In particular, the invention relates to thioxanthone derivative photoinitiator suitable to be in one drop filling process for manufacturing liquid crystal display device without the concern of liquid crystal contamination.

### TECHNICAL FIELD

This invention relates to a thioxanthone derivative photoinitiator, a radiation curable composition comprising the same and the use thereof. In particular, the invention relates to thioxanthone derivative photoinitiator suitable to be used in one drop filling (ODF) process for manufacturing liquid crystal display device without the concern of liquid crystal contamination.

### BACKGROUND OF THE INVENTION

Liquid crystal display (LCD) panels having the characteristics of being light-weight and high-definition have been widely used as display panels for a variety of apparatuses including cell phones and TVs. Conventionally, the process for producing a LCD panel is called a one-drop-filling (ODF) process comprising applying a sealant on a substrate having an electrode pattern and an alignment film under vacuum condition, dropping liquid crystal (LC) on the substrate having the sealant applied thereon, joining opposite facing substrates to each other under vacuum, then releasing the vacuum and performing ultraviolet (UV) irradiation or UV irradiation plus heating to cure the sealant and thereby producing a LCD cell.

Recently, development of LCD has been more towards the direction of “slim border” or “narrow bezel” design. Among several ways to achieve this goal, one is the use of a narrow width of the sealant. However, a thinner line of sealant creates more challenge with typical ODF process due to the fact that the process needs to meet very high reliability to prevent the liquid crystal material from contamination. The requirement of low contamination is particularly critical to ODF sealant products. No contamination is required to occur before and after the radiation curing process. The photoinitiator contained in the sealant composition should be stable before curing and generates little fragments during the curing.

On the other hand, although irradiating UV light from array side is also conceivable, challenges still remain since metal wirings and transistors on the array substrate overlap with the sealant pattern and create shadow area, which may in turn result in “shadow cure” issue as uncured portion of the sealant is apt to elute from sealant and comes into contact with LC which will also cause LC contamination. Shadow cure problem has hardly been solved for most sealant formulations for ODF process. The currently common practice is to use a side cure process in which light penetration depth can be a limiting factor.

A visible light curing process has drawn a lot of attention to solve the curing issue in ODF process. Oxime, thioxanthone and acylphosphineoxide types of photoinitiator are potential candidates to be used in ODF sealants. However, all these conventional photoinitiators have issues in the curing process. For examples, conventional photoinitiators based on thioxanthone and oxime have shown low curing speed and efficiency, while the acylphosphineoxide type has shown to introduce impurities by generate a large number of fragments in the curing process.

Thus, there is still a need for a thioxanthone type photoinitiator that can improve curing speed to solve the shadow cure issue and eliminate the liquid crystal contamination. In addition, the thioxanthone type photoinitiator should have excellent compatibility with the resin matrix.

### SUMMARY OF THE INVENTION

The present invention provides a thioxanthone derivative photoinitiator, represented by general formula (1):

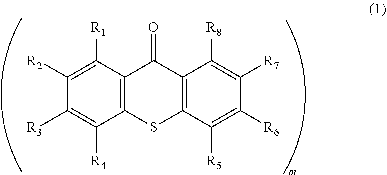

wherein m is 1 or 2;

R₁ to R8 each independently represents hydrogen, halogen, an optionally substituted alkyl group, an optionally substituted alkenyl group, an optionally substituted alkoxy group, an optionally substituted aralkyl group, an optionally substituted aryl group, or an optionally substituted heteroaryl group, and at least one of R₁ to R₈ represents a moiety of formula (2),

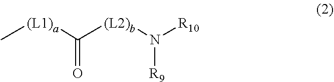

wherein L1 and L2 each independently represents an optionally substituted alkylene group, or an optionally substituted -Y-alkylene group, wherein the alkylene chain is optionally interrupted by —N(R₁₁)—, —O—, —S—, —S(O)—, —S(O)₂—, —C(O)—, —C(O)O—, —C(O)N(R₁₁)—, —S(O)₂N(R₁₁)—, —OC(O)N(R₁₁)—, —N(R₁₁)C(O)—, —N(R₁₁)SO₂—, —N(R₁₁)C(O)O—, —N(R₁₁)C(O)N(R₁₁)—, —N(R₁₁)S(O)₂N(R₁₁)—, —OC(O)—, or —C(O)N(R₁₁)—O—, in which R₁₁ is hydrogen or an optionally substituted C₁₋₁₂ aliphatic group, and Y represents an optionally substituted heteroatom;

a, b is 0, 1 or 2; and

R₉ and R₁₀ each independently represents an optionally substituted alkyl group, an optionally substituted alkoxyl group, an optionally substituted alkenyl group, an optionally substituted alkynyl group, an optionally substituted aralkyl group, an optionally substituted aryl group, or an optionally substituted heteroaryl group, or R₉ and R₁₀ taken together may form a 5-to 8-membered carbocyclic or hetero atom-containing ring.

The present invention also provides a radiation curable composition comprising the thioxanthone derivative photoinitiator according to the present invention, and the cured product obtained from the radiation curable composition.

Furthermore, the present invention provides a method of bonding materials together which comprises applying the radiation curable composition according to the present invention in a liquid form to a first substrate, bringing a second substrate in contact with the radiation curable composition applied to the first substrate, and subjecting the radiation curable composition to photo irradiation which will allow the radiation curable composition to cure to a solid form.

### BRIEF DESCRIPTION OF THE FIGURES

FIG. 1 illustrates a microscope photo of the curing depth in shadow cure test by UV light curing of the sealant composition containing the photoinitiator of Example 3.

FIG. 2 illustrates an enlarged view of the square frame in FIG. 1.

### DETAILED DESCRIPTION OF THE INVENTION

In the following passages the present invention is described in more detail. Each aspect so described may be combined with any other aspect or aspects unless clearly indicated to the contrary. In particular, any feature indicated as being preferred or advantageous may be combined with any other feature or features indicated as being preferred or advantageous.

In the context of the present invention, the terms used are to be construed in accordance with the following definitions, unless a context dictates otherwise.

As used herein, the singular forms “a”, “an” and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or process steps.

The recitation of numerical end points includes all numbers and fractions subsumed within the respective ranges, as well as the recited end points.

All references cited in the present specification are hereby incorporated by reference in their entirety.

Unless otherwise defined, all terms used in the disclosing the invention, including technical and scientific terms, have the meaning as commonly understood by one of the ordinary skill in the art to which this invention belongs to. By means of further guidance, term definitions are included to better appreciate the teaching of the present invention.

In one aspect, the present invention provides a thioxanthone derivative photoinitiator, represented by general formula (1):

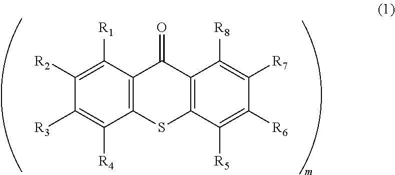

wherein m is 1 or 2;

R₁ to R₈ each independently represents hydrogen, halogen, an optionally substituted alkyl group, an optionally substituted alkenyl group, an optionally substituted alkoxy group, an optionally substituted aralkyl group, an optionally substituted aryl group, or an optionally substituted heteroaryl group, and at least one of R₁ to R₈ represents a moiety of formula (2),

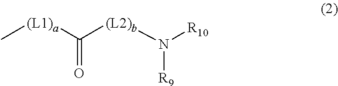

wherein L1 and L2 each independently represents an optionally substituted alkylene group, or an optionally substituted —Y-alkylene group, wherein the alkylene chain is optionally interrupted by —N(R₁₀)—, —O—, —S—, —S(O)—, —S(O)₂—, —C(O)—, —C(O)O—, —C(O)N(R₁₁)—, —S(O)₂N(R₁₁)—, —OC(O)N(R₁₁)—, —N(R₁₁)C(O)—, —N(R₁₁)SO₂—, —N(R₁₁)C(O)O—, —N(R₁₁)C(O)N(R₁₁)—, —N(R₁₁)S(O)₂N(R₁₁)—, —OC(O)—, or —C(O)N(R₁₁)—O—, in which R₁₁ is hydrogen or an optionally substituted C₁₋₁₂ aliphatic group, and Y represents an optionally substituted heteroatom;

a, b is 0, 1 or 2; and

R₉ and R₁₀ each independently represents an optionally substituted alkyl group, an optionally substituted alkoxyl group, an optionally substituted alkenyl group, an optionally substituted alkynyl group, an optionally substituted aralkyl group, an optionally substituted aryl group, or an optionally substituted heteroaryl group, or R₉ and R₁₀ taken together may form a 5- to 8-membered carbocyclic or hetero atom-containing ring.

It has been surprisingly found that the thioxanthone derivative photoinitiator according to the present invention provides an improved curing speed to a radiation curable sealant even in dark region and thus is suitable for the use in ODF process. In addition, the thioxanthone derivative photoinitiator is well compatible with resin matrix and may shorten the pretreatment of the sealant composition.

Thioxanthone derivative photoinitiators of the present disclosure include those described generally for formula (I), above, and are further illustrated by the classes, subclasses, and species disclosed herein. It will be appreciated that some subsets described for each variable herein can be used for any of the structural subsets as well. As used herein, the following definitions shall apply unless otherwise indicated.

As described herein, thioxanthone derivative photoinitiators of the present disclosure may be optionally substituted with one or more substituents, such as are disclosed generally above, or as exemplified by particular classes, subclasses, and species disclosed herein. It will be appreciated that the phrase “optionally substituted” is used interchangeably with the phrase “substituted or unsubstituted.” In general, the term “substituted”, whether preceded by the term “optionally” or not, means that a hydrogen radical of the designated moiety is replaced with the radical of a specified substituent, provided that the substitution results in a stable or chemically feasible compound. The term “substitutable”, when used in reference to a designated atom, means that attached to the atom is a hydrogen radical, which hydrogen atom can be replaced with the radical of a suitable substituent. Unless otherwise indicated, an “optionally substituted” group may have a substituent at each substitutable position of the group, and when more than one position in any given structure may be substituted with more than one substituent selected from a specified group, the substituent may be either the same or different at every position. Combinations of substituents envisioned by this disclosure are for instance, those that result in the formation of stable or chemically feasible compounds.

The term “aliphatic” or “aliphatic group”, as used herein, means an optionally substituted straight-chain or branched C₁₋₁₂ hydrocarbon, or a cyclic C₁₋₁₂ hydrocarbon which is completely saturated or which contains one or more units of unsaturation, but which is not aromatic. For example, suitable aliphatic groups include optionally substituted linear, branched or cyclic alkyl, alkenyl, alkynyl groups and hybrids thereof, such as (cycloalkyl)alkyl, (cycloalkenyl)alkyl, or (cycloalkyl)alkenyl. Unless otherwise specified, in various embodiments, aliphatic groups have 1-12, 1-10, 1-8, 1-6, 1-5, 1-4, 1-3, or 1-2 carbon atoms.

The term “alkyl” or “alkylene”, used alone or as part of a larger moiety, refers to an optionally substituted straight or branched chain hydrocarbon group having 1-12, 1-10, 1-8, 1-6, 1-5, 1-4, 1-3, or 1-2 carbon atoms.

The term “alkenyl”, used alone or as part of a larger moiety, refers to an optionally substituted straight or branched chain hydrocarbon group having at least one double bond and having 2-12, 2-10, 2-8, 2-6, 2-5, 2-4, or 2-3 carbon atoms.

The term “cycloalkyl” refers to an optionally substituted saturated ring system of about 3 to about 10 ring carbon atoms. Exemplary monocyclic cycloalkyl rings include cyclopropyl, cyclobutyl, cyclopentyl, cyclohexyl, and cycloheptyl.

As used herein, the term “halogen” or “halo” means F, Cl, Br, or I.

The term “heteroatom” refers to one or more of oxygen, sulfur, nitrogen, phosphorus, or silicon (including, any oxidized form of nitrogen, sulfur, phosphorus, or silicon; the quatemized form of any basic nitrogen or; a substitutable nitrogen of a heterocyclic ring, for example N, NH or NR+).

The term “aryl”, used alone or as part of a larger moiety, e.g., “aralkyl”, “aralkoxy”, or “aryloxyalkyl”, refer to an optionally substituted C₆₋₁₄ aromatic hydrocarbon moiety comprising one to three aromatic rings. In at least one embodiment, the aryl group is a C₆₋₁₀ aryl group. Aryl groups include, without limitation, optionally substituted phenyl, naphthyl, or anthracenyl. The term “aryl”, as used herein, also include groups in which an aryl ring is fused to one or more cycloaliphatic rings to form an optionally substituted cyclic structure such as a tetrahydronaphthyl, indenyl, or indanyl ring.

The term “aralkyl” refers to an aryl group covalently attached to an alkyl group, either of which independently is optionally substituted. In at least one embodiment, the aralkyl group is C₆₋₁₀ aryl C₁₋₁₂ alkyl, including, without limitation, benzyl, phenethyl, and naphthylmethyl.

The term “heteroaryl”, used alone or as part of a larger moiety, e.g., “heteroaralkyl”, or “heteroaralkoxy”, refer to groups having 5 to 14 ring atoms, such as 5, 6, 9, or 10 ring atoms; having 6, 10, or 14 π electrons shared in a cyclic array; and having, in addition to carbon atoms, from one to five heteroatoms. A heteroaryl group may be mono-, bi-, tri-, or polycyclic, for instance mono-, bi-, or tricyclic, such as mono- or bicyclic. The term “heteroatom” refers to nitrogen, oxygen, or sulfur, and includes any oxidized form of nitrogen or sulfur, and any quatemized form of a basic nitrogen. For example, a nitrogen atom of a heteroaryl may be a basic nitrogen atom and may also be optionally oxidized to the corresponding N-oxide. The term “heteroaryl”, as used herein, also include groups in which a heteroaromatic ring is fused to one or more aryl, cycloaliphatic, or heterocycloaliphatic rings. Nonlimiting examples of heteroaryl groups include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl. The term “heteroaralkyl” refers to an alkyl group substituted by a heteroaryl, wherein the alkyl and heteroaryl portions independently are optionally substituted.

In some embodiments of the present invention, m is 2. In some embodiments of the present invention, m is 1.

In some embodiments of the present invention, any of R₅ to R₈ represents a moiety of formula (2), and the others of R₁ to R₈ each independently represents hydrogen, halogen, an alkyl group, an alkoxy group, an aralkyl group, an aryl group, or a heteroaryl group. In some preferred embodiments of the present invention, R₆ represents a moiety of formula (2), and R₁ to R₅, R₇ and R₈ each independently represents hydrogen, halogen, an alkyl group, an alkoxy group, an aralkyl group, an aryl group, or a heteroaryl group. In some preferred embodiments of the present invention, R₇ represents a moiety of formula (2), and the others of R₁ to R₆ and R₈ each independently represents hydrogen, halogen, an alkyl group, an alkoxy group, an aralkyl group, an aryl group, or a heteroaryl group. In some preferred embodiments of the present invention, R8 represents a moiety of formula (2), and R₁ to R₇ each independently represents hydrogen, halogen, an alkyl group, an alkoxy group, an aralkyl group, an aryl group, or a heteroaryl group.

In some embodiments of the present invention, R₉ and R₁₀ each independently represents an optionally substituted alkyl group, an optionally substituted alkenyl group, an optionally substituted alkynyl group, an optionally substituted aralkyl group, an optionally substituted aryl group, or an optionally substituted heteroaryl group.

In some embodiments of the present invention, R₉ and R₁₀ taken together may form a 5- to 8-membered, preferably 6-membered carbocyclic or hetero atom-containing ring.

In some embodiments of the present invention, R₉ and R₁₀ is not taken together to form a 5- to 8-membered carbocyclic or heteroatom-containing ring, and the thioxanthone derivative photoinitiator is represented by the formula (3):

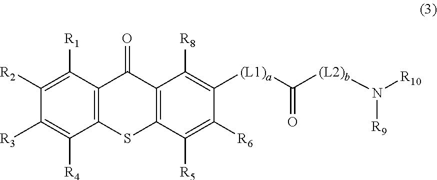

wherein R₁ to R₆, R₈ to R₁₀, L1, L2, a, and b are defined as for formula (1).

In some embodiments of the present invention, R₈ to R₉ taken together form a 5- or 6-member heteroatom-containing ring, and the thioxanthone derivative photoinitiator is represented by the formula (4):

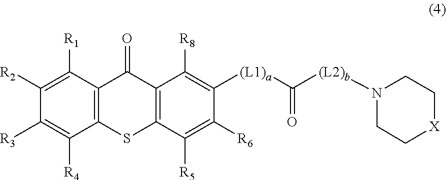

wherein R₁ to R₆, R₈, L1, L2, a, and b are defined as above, X represents hydrogen or an optionally substituted heteroatom.

Unless otherwise stated, the following values are described for any of formulas (1) to (4).

In some embodiments, other than the group(s) represented by the moiety of formula (2), the rest of R₁ to R₈ each independently represents hydrogen, halogen, an optionally substituted C₁₋₈ alkyl group, an optionally substituted C₂₋₈ alkenyl group, an optionally C₁₋₈ substituted alkoxy group, an optionally substituted C₇₋₁₄ aralkyl group, an optionally substituted C₆₋₁₄ aryl group, or an optionally substituted C₅₋₁₃ heteroaryl group. In some embodiments, other than the group(s) represented by the moiety of formula (2), the rest of R₁ to R₈ each independently is hydrogen, halogen, C₁₋₆ alkyl group, or C₁₋₆ alkoxy group. In some embodiments, other than the group(s) represented by moiety of formula (2), the rest of R₁ to R₈ each independently is hydrogen, C₁₋₆ alkyl group, or C₁₋₆ alkoxy group. In some embodiments, other than the group(s) represented by the moiety of formula (2), in the rest of R₁ to R₈, one group is C₁₋₆ alkyl group or C₁₋₆ alkoxy group, and others are hydrogen. In some embodiments, other than the group(s) represented by the moiety of formula (2), in the rest of R₁ to R₈, two or more groups are C₁₋₆ alkyl group or C₁₋₆ alkoxy, and others are hydrogen. In some embodiments, other than the group(s) represented by the moiety of formula (2), the rest of R₁ to R₈ are hydrogen.

In some embodiments, a is 0, and b is 0 or 1. In some embodiments, when a is not 0, L1 represents an optionally substituted C₁₋₆ alkylene group, wherein the alkylene chain optionally is interrupted by —N(R₁₁)—, —O—, —S—, —S(O)—, —S(O)₂—, —C(O)—, —C(O)O—, —C(O)N(R₁₁)—, —S(O)₂N(R₁₁)—, —OC(O)N(R₁₁)—, —N(R₁₁)C(O)—, —N(R₁₁)SO₂—, —N(R₁₁)C(O)O—, —N(R₁₁)C(O)N(R₁₁)—, —N(R₁₁)S(O)₂N(R₁₁)—, —OC(O)—, or —C(O)N(R₁₁)—O—, wherein R₁₁ is hydrogen or an optionally substituted C₁₋₆ aliphatic group. In some embodiments, when a is not 0, L1 represents an optionally substituted C₁₋₆ alkylene group, wherein the alkylene chain is optionally interrupted by —N(R₁₁)—, —O—, —S—, —S(O)—, —S(O)₂—, —C(O)—, or —C(O)O—, wherein R₁₁ is hydrogen or an optionally substituted C₁₋₆ aliphatic group. In some embodiments, when a is not 0, L1 represents an optionally substituted C₁₋₆ alkylene group, wherein the alkylene chain is optionally interrupted by —O—, —S—, —S(O)—, —S(O)₂—, —C(O)—, or —C(O)O—. In some embodiments, when a is not 0, L1 represents a C₁₋₆ alkylene group, wherein the alkylene chain optionally is interrupted by —O—, —C(O)—, or —C(O)O—. In some embodiments, when a is not 0, L1 represents an unsubstituted and uninterrupted C₁₋₆ alkylene group, preferably is methylene or ethylene. In some embodiments, when a is not 0, L1 represents an optionally substituted C₁₋₆ alkoxylene group. In some embodiments, when a is not 0, L1 represents an unsubstituted C₁₋₆ alkoxylene group. In some embodiments, when a is not 0, L1 represents methoxylene or ethoxylene. In some embodiments, when a is not 0, L1 represents an —X-alkylene group, wherein the alkylene chain is optionally interrupted by —N(R₁₀)—, —O—, —S—, —S(O)—, —S(O)₂—, —C(O)—, —C(O)O—, —C(O)N(R₁₁)—, —S(O)₂N(R₁₁)—, —OC(O)N(R₁₁)—, —N(R₁₁)C(O)—, —N(R₁₁)SO₂—, —N(R₁₁)C(O)O—, —N(R₁₁)C(O)N(R₁₁)—, —N(R₁₁)S(O)₂N(R₁₁)—, —OC(O)—, or —C(O)N(R₁₁)—O—, in which R₁₁ is hydrogen or an optionally substituted C₁₋₄ aliphatic group, and X represents an optionally substituted heteroatom, such as O, S, NH, or NR₁₂, wherein R₁₂ is a C₁₋₆ alkyl group or C₁₋₆ alkoxyl group.

In some embodiments, when b is not 0, L2 represents an optionally substituted C₁₋₆ alkylene group, wherein the alkylene chain optionally is interrupted by —N(R₁₁)—, —O—, —S—, —S(O)—, —S(O)₂—, —C(O)—, —C(O)O—, —C(O)N(R₁₁)—, —S(O)₂N(R₁₀)—, —OC(O)N(R₁₁)—, —N(R₁₁)C(O)—, —N(R₁₁)SO₂—, —N(R₁₁)C(O)O—, —N(R₁₁)C(O)N(R₁₁)—, N(R₁₁)S(O)₂N(R₁₁)—, —OC(O)—, or —C(O)N(R₁₁)—O—, wherein R₁₀ is hydrogen or an optionally substituted C₁₋₆ aliphatic group. In some embodiments, when b is not 0, L2 represents an optionally substituted C₁₋₆ alkylene group, wherein the alkylene chain optionally is interrupted by —N(R₁₀)—, —O—, —S—, —S(O)—, —S(O)₂—, —C(O)—, or —C(O)O—, wherein R₁₀ is hydrogen or an optionally substituted C₁₋₆ aliphatic group. In some embodiments, when b is not 0, L2 represents an optionally substituted C₁₋₆ alkylene group, wherein the alkylene chain optionally is interrupted by —O—, —S—, —S(O)—, —S(O)₂—, —C(O)—, or —C(O)O—. In some embodiments, when b is not 0, L2 represents an C₁₋₆ alkylene group, wherein the alkylene chain optionally is interrupted by —O—, —C(O)—, or —C(O)O—. In some embodiments, when b is not 0, L2 represents an unsubstituted and uninterrupted C₁₋₆ alkylene group, preferably is methylene or ethylene. In some embodiments, when b is not 0, L2 represents an optionally substituted C₁₋₆ alkoxylene group. In some embodiments, when when b is not 0, L2 represents an optionally substituted C₁₋₆ alkoxylene group. In some embodiments, when when b is not 0, L2 represents methoxylene or ethoxylene. In some embodiments, when b is not 0, L2 represents an —Y-alkylene group, wherein the alkylene chain is optionally interrupted by —N(R₁₀)—, —O—, —S—, —S(O)—, —S(O)₂—, —C(O)—, —C(O)O—, —C(O)N(R₁₁)—, —S(O)₂N(R₁₁)—, —OC(O)N(R₁₁)—, —N(R₁₁)C(O)—, —N(R₁₁)SO₂—, —N(R₁₁)C(O)O—, —N(R₁₁)C(O)N(R₁₁)—, —N(R₁₁)S(O)₂N(R₁₁)—, —OC(O)—, or —C(O)N(R₁₁)—O—, in which R₁₁ is hydrogen or an optionally substituted C₁₋₄ aliphatic group, and Y represents an optionally substituted heteroatom, such as O, S, NH, or NR₁₂, wherein R₁₂ is a C₁₋₆ alkyl group or C₁₋₆ alkoxyl group.

In some embodiments, R₉ and R₁₀ each independently represents an optionally substituted C₁₋₁₂ alkyl group, an optionally substituted C₁₋₁₂alkoxyl group, an optionally substituted C₂₋₁₂ alkenyl group, an optionally substituted C₃₋₁₂alkynyl group, an optionally substituted C₇₋₁₁ aralkyl group, an optionally substituted C₆₋₁₀ aryl group, or an optionally substituted C₅₋₉ heteroaryl group. In some embodiments, R₉ and R₁₀ each represents a C₁₋₁₂ alkyl group or C₁₋₁₂ alkoxyl group. In some embodiments, R₉ and R₁₀ each independently represents a C₁₋₆ alkyl group, such as methyl, ethyl, n-propyl, i-propyl, n-butyl (-Bu), i-butyl, or t-butyl.

In some embodiments, according to formula (4), X is optionally substituted, preferably alkyl substituted nitrogen, oxygen or sulfur. In some embodiments, according to formula (4), X is NH, S or O. In some embodiments, according to formula (4), R₁ to R₆ and R₈ all represent hydrogen, a is 0 or 1, b is 0, L1 is C₁₋₆ alkylene group and X is NH, S or O. In some embodiments, according to formula (4), R₁ to R₆ and R₈ all represent hydrogen, a is 0, b is 0, and X is S or O. In some embodiments, according to formula (4), R₁ to R₆ and R₈ all represent hydrogen, a is 1, b is 0, L1 is methylene or ethylene, and X is oxygen or sulfur.

In one preferred embodiment, R₇ represents a moiety of formula (2), R₁ to R₆ and R₈ are all hydrogen, a=b=0, R₉ and R₁₀ are n-butyl.

In another preferred embodiment, R₇ represents a moiety of formula (2), R₁ to R₆ and R₈ are all hydrogen, a=b=0, R₉ and R₁₀ taken together form a 6-member heteroatom-containing ring, and X is O.

In yet another preferred embodiment, R₇ represents a moiety of formula (2), R₁ to R₆ and R₈ are all hydrogen, a=b=0, R₉ and R₁₀ taken together form a 6-member heteroatom-containing ring, and X is S.

In yet another preferred embodiment, R₇ represents a moiety of formula (2), R₁ to R₆ and R₈ are all hydrogen, a=0, b=1, L2 is ethoxylene, and R₉ and R₁₀ are both ethyl.

In yet another preferred embodiment, R₇ represents a moiety of formula (2), R₁ to R₆ and R₈ are all hydrogen, a=1, b=0, L1 is methylene, and R₉ and R₁₀ are n-butyl.

In yet another preferred embodiment, R₇ represents a moiety of formula (2), R₁ to R₆ and R₈ are all hydrogen, a=1, b=0, L1 is n-propylene, and R₉ and R₁₀ are n-butyl.

In yet another preferred embodiment, R₇ represents a moiety of formula (2), R₁ to R₆ and R₈ are all hydrogen, a=1, b=0, L1 is methylene, R₉ and R₁₀ taken together form a 6-member heteroatom-containing ring, and X is N—CH₃.

In yet another preferred embodiment, R₇ represents a moiety of formula (2), R₁ to R₆ and R₈ are all hydrogen, a=1, b=0, L1 is methylene, R₉ and R₁₀ taken together form a 6-member heteroatom-containing ring, and X is S.

In yet another preferred embodiment, R₇ represents a moiety of formula (2), R₁ to R6 and R8 are all hydrogen, a=1, b=0, L1 is methylene, R₉ and R₁₀ taken together form a 6-member heteroatom-containing ring, and X is O.

In yet another preferred embodiment, R₇ represents a moiety of formula (2), R₂ is methyl, R₁, R₃ to R₆ and R₈ are all hydrogen, a=b=0, and R₉ and R₁₀ are n-butyl.

In yet another preferred embodiment, R₇ represents a moiety of formula (2), R₃ is isopropyl, R₁, R₂, R₄ to R₆ and R₈ are all hydrogen, a=b=0, and R₉ and R₁₀ are n-butyl.

In yet another preferred embodiment, R₇ represents a moiety of formula (2), R₁ is Cl, R₁, R₂, R₄ to R₆ and R₈ are all hydrogen, a=b=0, and R₉ and R₁₀ are n-butyl.

In yet another preferred embodiment, R₇ represents a moiety of formula (2), R₂ is methoxyl, R₁, R₃ to R₆ and R₈ are all hydrogen, a=b=0, and R₉ and R₁₀ are n-butyl.

In yet another preferred embodiment, R₆ represents a moiety of formula (2), R₁ to R₅, R₇ and R₈ are all hydrogen, a=b=0, and R₉ and R₁₀ are n-butyl.

In yet another preferred embodiment, R₈ represents a moiety of formula (2), R₁ to R₇ are all hydrogen, a=b=0, and R₉ and R₁₀ are n-butyl.

In yet another preferred embodiment, R₇ represents a moiety of formula (2), R₁ to R₆ and R₈ are all hydrogen, a=0, b=1, L2 is —S-ethylene, and R₉ and R₁₀ are both ethyl.

In yet another preferred embodiment, R₇ represents a moiety of formula (2), R₁ to R₆ and R₈ are all hydrogen, a=0, b=1, L2 is —NH-ethylene, and R₉ and R₁₀ are both ethyl.

In yet another preferred embodiment, R₇ represents a moiety of formula (2), R₁ to R₆ and R₈ are all hydrogen, a=b=0, and R₉ and R₁₀ taken together form a 6-member heteroatom-containing ring, and X is N—CH₃.

In yet another preferred embodiment, R₇ represents a moiety of formula (2), R₁ to R₆ and R₈ are all hydrogen, a=0, b=1, L2 is —NH-ethylene, and R₉ and R₁₀ taken together form a 6-member heteroatom-containing ring, and X is H.

In yet another preferred embodiment, R₇ represents a moiety of formula (2), R₁ to R₆ and R₈ are all hydrogen, a=0, b=1, L2 is —NH-ethylene, and R₉ and R₁₀ taken together form a 6-member heteroatom-containing ring, and X is S.

In some preferred embodiments, the thioxanthone derivative photoinitiators according to the present invention are selected from the group consisting of:

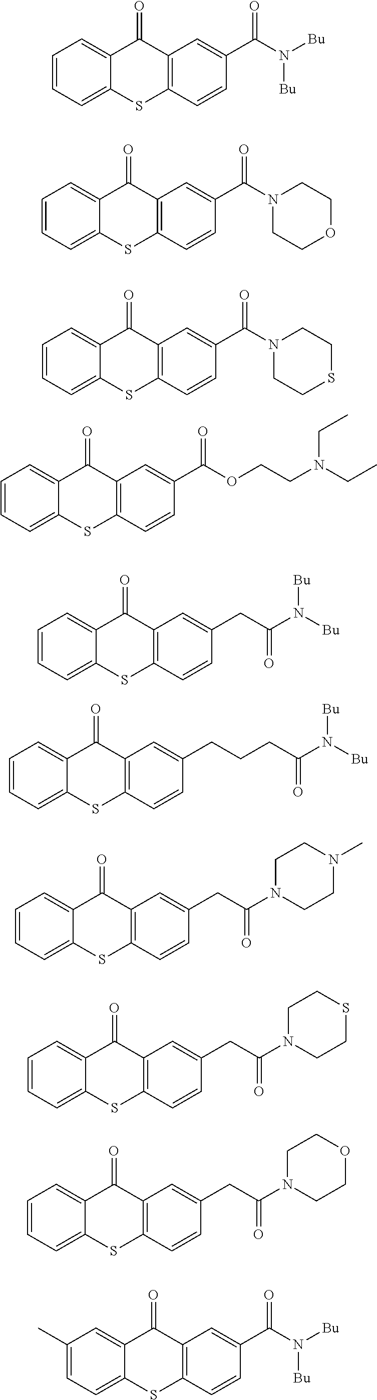

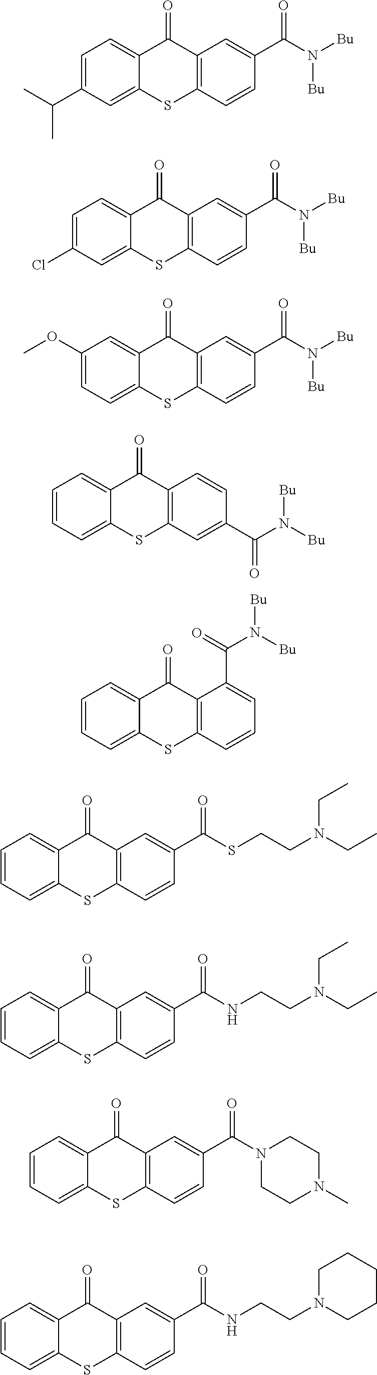

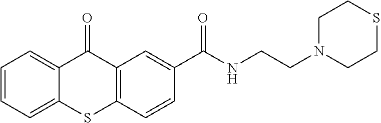 [SMILES: O=C(NCCN1CCSCC1)c1ccc2sc3ccccc3c(=O)c2c1]

It has been surprisingly found that the thioxanthone derivative photoinitiators according to the present invention possess high photoinitiation efficiency and thus contribute to a better curing speed of the radiation curable composition comprising the thioxanthone derivative photoinitiators.

Not binding by any theory, it is believed that tertiary amine structure introduced as tertiary amide or tertiary amine into the thioxanthone photoinitiator conjugates with the thioxanthone structure. This results in the shifting of the absorption spectrum of the photoinitiator to a longer wavelength. In addition, the linking chain such as the alkane chain or ring structure may initiate an intramolecular chain transfer effect in the process of photoinitiation, and the fragmentation of the photoinitiator in the curing process could be significantly reduced, and in turn improves the curing speed of the radiation curable composition and suppresses the oxygen inhibition.

In another aspect, the present invention provides a radiation curable composition comprising the thioxanthone derivative photoinitiator according to present invention.

In addition to the thioxanthone derivative photoinitiator, the radiation curable composition may comprise radiation curable resin component and a latent curing agent.

According to the present invention, the amount of the thioxanthone derivative photoinitiator used in the composition is in an amount of 0.1 to 5% by weight, and preferably 0.5 to 1% by weight, based on the total amount of composition.

The radiation curable resin component can be selected from (meth)acrylate resin, bismaleimide resin, and mixture thereof.

The (meth)acrylate resin includes but not limited to 2-hydroxyethyl acrylate, 2-hydroxypropyl(meth)acrylate, 4-hydroxybutyl(meth)acrylate, 2-hydroxybutyl (meth)acrylate, isobutyl (meth)acrylate, t-butyl (meth)acrylate, isooctyl (meth)acrylate, lau-ryl (meth)acrylate, stearyl (meth)acrylate, isobornyl (meth)acrylate, cyclohexyl (meth)acrylate, 2-methoxyethyl (meth)acrylate, menubutoxy ethylene glycol (meth)acrylate, 2-ethoxyethyl (meth)acrylate, tetrahydrofurfuryl (meth)acrylate, benzyl (meth)acrylate, ethyl carbitol (meth)acrylate, phenoxyethyl (meth)acrylate, carboxymethyl diethylene glycol (meth)acrylate, phenoxy polyethylene glycol (meth)acrylate, methoxypolyethylene glycol (meth)acrylate, 2,2,2,-trifluoroethyl (meth)acrylate, 2,2,3,3,-tetrafuruo(meth)acrylate, 1 H,1H,5H-octafluoropentyl (meth)acrylate, imide (meth)acrylate, methyl (meth)acrylate, ethyl (meth)acrylate, n-butyl (meth)acrylate, propyl (meth)acrylate, n-butyl (meth)acrylate, cyclohexyl (meth)acrylate, 2-ethylhexyl (meth)acrylate, n-octyl (meth)acrylate, isononyl (meth)acrylate, isomyristyl (meth)acrylate, 2-butoxyethyl (meth)acrylate, 2-phenoxyethyl (meth)acrylate, bicyclopentenyl (meth)acrylate, isodecyl (meth)acrylate, diethylaminoethyl (meth)acrylate, dimethylaminoethyl (meth)acrylate, 2-(meth)acryloyl acid, 2-(meth)acryloyloxyethyl hexahydrophthalic acid, 2-(meth)acryloyloxyethyl 2-hydroxypropyl phthalate, glycidyl (meth)acrylate, 2-(meth)acryloyloxyethyl phosphate, 1,4-butanediol di(meth)acrylate, 1,3-butanediol di(meth)acrylate, 1,6-hexanediol di(meth)acrylate, 1,9-nonanediol di(meth)acrylate 1,10-decanediol di(meth)acrylate, 2-n-butyl-2-ethyl -1,3-propanediol di(meth)acrylate, dipropylene glycol di (meth)acrylate, tripropylene glycol di(meth)acrylate, polypropylene glycidyl(meth)acrylate, ethylene glycol di(meth)acrylate, diethyleneglycol di(meth)acrylate, tetra-ethylene glycol di(meth)acrylate, polyethylene glycol di(meth)acrylate, propylene oxide addition bisphenolLumpur A di(meth)acrylate, ethylene oxide addition bi-sphenol A di(meth)acrylateRelate, ethylene oxide addition bisphenol F di(meth)acrylate, cyclopentadiene distearate(meth)acrylate, 1,3-butylene glycol di(meth)acrylate, neopentyl glycol di(meth)acrylate, ethylene oxide modified isocyanuric acid di(meth)acrylate, 2-hydroxy-3-(meth)acryloyl Rokishipuropiru(meth)acrylate, carbonate diol di(meth)acrylate, pentaerythritol tri(meth)acrylate, trimethylolpro-pane tri(meth)acrylate, propylene oxide adduct of trimethylolpropane tri(meth)acry-late Bets ethylene oxide adduct of trimethylolpropane tri(meth)acrylate, Kapurora Lactone-modified trimethylolpropane tri(meth)acrylate, ethylene oxide addition Lee Soshianuru acid tri(meth)acrylate, dipentaerythritol penta(meth)acrylate, dipentae-rythritol hexa(meth)acrylate, ditrimethylolpropane propNtetora(meth)acrylate, pen-taerythritol tetra(meth)acrylate, grayed Riserintori(meth)acrylate, propylene oxide addition glycerin tri(meth)acrylate, tris(meth)acryloyloxyethyl phosphate, and the like. Of these, those having an aromatic ring (meth)acrylate compound can be preferably used. Examples of commercially available compounds are, for example, EB230, EB264, EB265, EB284, EB280, EB1290, EB270, EB4833, EB8210, EB8402, EB8808(aliphatic urethane acrylate), EB220, EB4827, EB4849 (aromatic urethane acrylate), EB657, EB885, EB600, EB3200, EB3700, EB3702, EB3703, EB3720 (Daicel Cytec Co., Ltd.); CN8000NS, CN8001NS, CN8003, CN9001NS, CN9002, CN9014NS, CN991NS (urethane acrylate), CN146, CN2203NS, CN2254NS, CN2261, CN2302, CN293, CN3108, CN704, CN8200 (polyester acrylate), CN104NS, CN120NS, CN131NS, CN2003NS, CN159 (epoxy acrylate)(Sartomer) etc.

The maleimide resin includes those having the generic structure:

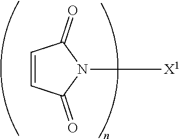

in which n is 1 to 3 and X¹ is an aliphatic or aromatic group. Exemplary X¹ entities include, poly(butadienes), poly(carbonates), poly(urethanes), poly(ethers), poly(esters), simple hydrocarbons, and simple hydrocarbons containing functionalities such as carbonyl, carboxyl, amide, carbamate, urea, ester, or ether. These types of resins are commercially available and can be obtained, for example, from Dainippon Ink and Chemical, Inc. Exemplary embodiments include, but not limited to:

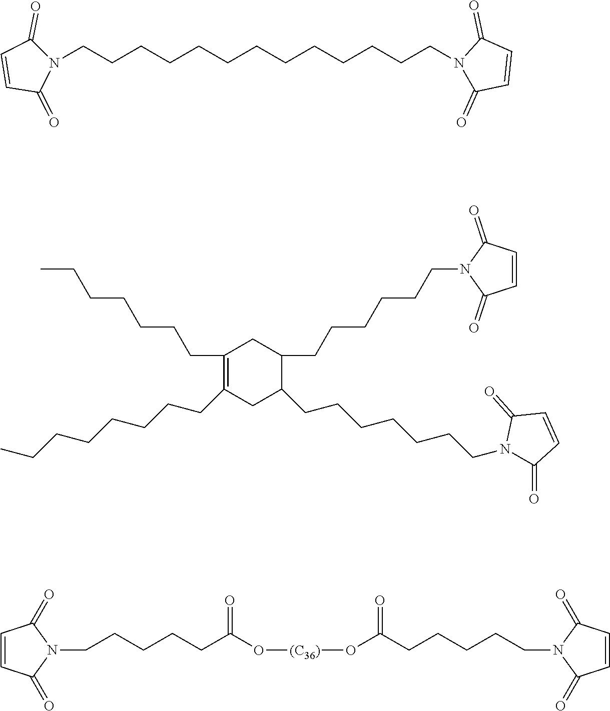

in which C36 represents a linear or branched hydrocarbon chain (with or without cyclic moieties) of 36 carbon atoms;

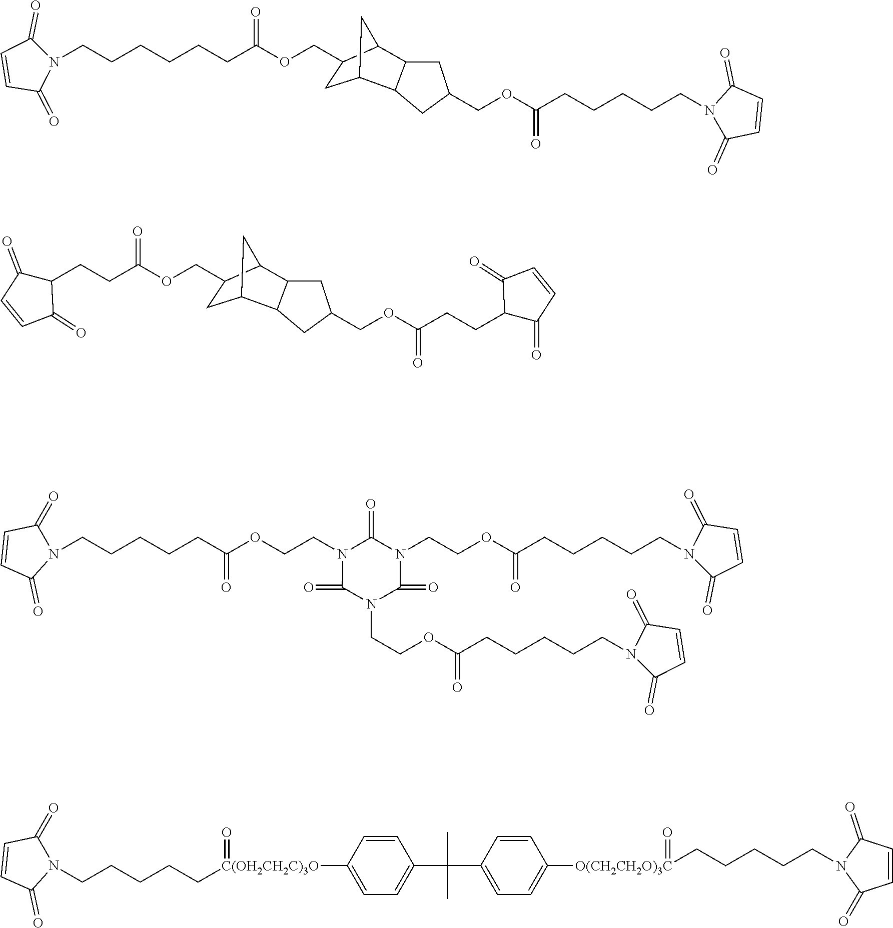

According to the present invention, the amount of the radiation curable resin component used in the composition is in an amount of 30 to 90% by weight, and preferably 50 to 80% by weight, based on the total amount of composition.

The latent curing agent is used to cure the radiation curable composition when heated. It can be obtained easily from the commercially available latent curing agent and used alone or in a combination of two or more kinds.

Specifically, the latent curing agent to be preferably used includes amine-based compounds, fine-powder-type modified amine and modified imidazole based com-pounds. Examples of the amine-based latent curing agent include dicyandiamide, hydrazides such as adipic acid dihydrazide, oxalic acid dihydrazide, malonic acid dihydrazide, succinic acid dihydrazide, glutaric acid dihydrazide, suberic acid dihydrazide, azelaic acid dihydrazide, sebacic acid dihydrazide, and phthalic acid dihydrazide. The modified amine and modified imidazole based compounds include core-shell type in which the surface of an amine compound (or amine adducts) core is coated with the shell of a modified amine product (surface adduction and the like) and master-batch type hardeners as a blend of the core-shell type curing agent with an epoxy resin. These types of latent curing agents are capable of providing a blend having good viscosity stability and can be cured at a relatively lower temperature (70-130° C.).

Examples of commercially available latent curing agents include, but not limited to Adeka Hardener EH-4357S (modified-amine-type), Adeka Hardener EH-4357PK (modified-amine-type), EH-5057PK (modified-amine-type), Adeka Hardener EH-4380S (special hybrid-type), Fujicure FXR-1081(modified-amine-type), Fujicure FXR-1020 (modified-amine-type), Sunmide LH-210 (modified-imidazole-type), Sun-mide LH-2102 (modified-imidazole-type), Sunmide LH-2100 (modified-imidazole-type), Ajicure PN-23 (modified-imidazole-type), Ajicure PN-F (modified-imidazole-type), Ajicure PN-23J (modified-imidazole-type), Ajicure PN-31 (modified-imidazole-type), Ajicure PN-31J (modified-imidazole-type), Novacure HX-3722 (master-batch type), Novacure HX-3742 (master-batch type), Novacure HX-3613 (master-batch type), and the like.

Latent curing agents having a melting temperature of 50 to 110° C., particularly having a melting temperature of 60° to 100° C. are preferred. Those having a melting temperature lower than 40° C. have the problem of poor viscosity stability, while those having a melting temperature higher than 120° C. need longer time of thermal cure, which causes a higher tendency of liquid crystal contamination.

The amount of the latent curing agent contained in the composition may be appropriately selected depending on the kind of the latent curing agent and the resin amount in the composition. Normally, the amount of the latent curing agent used in the composition is in an amount of 10 to 70% by weight, and preferably 20 to 50% by weight, based on the total amount of composition.

The component that may be contained in the composition as optional includes, for example, thermoplastic polymer, organic or inorganic filler, thixotropic agent, silane coupling agent, diluent, modifier, coloring agent such as pigment and dye, surfactant, preservative, stabilizer, plasticizer, lubricant, defoamer, leveling agent and the like; however it is not limited to these. In particular, the composition preferably comprises an additive selected from the group consisting of organic or inorganic filler, a thixotropic agent, and a silane coupling agent.

The filler includes, but not limited to, inorganic filler such as silica, diatomaceous earth, alumina, zinc oxide, iron oxide, magnesium oxide, tin oxide, titanium oxide, magnesium hydroxide, aluminium hydroxide, magnesium carbonate, barium sul-phate, gypsum, calcium silicate, talc, glass bead, sericite activated white earth, ben-tonite, aluminum nitride, silicon nitride, and the like; meanwhile, organic filler such as poly methyl methacrylate, poly ethyl methacrylate, poly propyl methacrylate, poly butyl methacrylate, butylacrylate-methacrylic acid-methyl methacrylate copolymer, poly acrylonitrile, polystyrene, poly butadiene, poly pentadiene, poly isoprene, poly isopropylene, and the like. These may be used alone or in combination thereof.

The thixotropic agent includes, but not limited to, talc, fume silica, superfine surface-treated calcium carbonate, fine particle alumina, plate-like alumina; layered compound such as montmorillonite, spicular compound such as aluminium borate whisker, and the like. Among them, talc, fume silica and fine alumina are preferred.

The silane coupling agent includes, but not limited to, γ-aminopropyltriethoxysilane, γ-mercaptopropyltrimethoxysilane, γ-methacryloxypropyltrimethoxysilane, γ-glycidoxypropyltrimethoxylsilane, and the like.

In one particular embodiment, the radiation curable composition of present invention comprises:

(1) about 30% to about 90%, preferably about 50% to about 80% by weight of a radiation curable resin component,

(2) about 10% to about 70%, preferably about 20% to about 50% by weight of a latent curing agent, and

(3) about 0.1% to about 5%, preferably about 0.5% to about 1% by weight of the thioxanthone derivative photoinitiator according to the present invention, in which the weight percentages are based on the total weight of the composition.

The radiation curable composition can be prepared by conventional methods. For example, the radiation curable composition is prepared by sufficiently mixing all components by a stirrer and then a three roll miller, under room temperature, to give a well distributed curable resin compositions.

In another aspect, the present invention also provides a method for bonding articles together which comprises applying the radiation curable composition according to the present invention in a liquid form to a first substrate, bringing a second substrate in contact with the radiation curable composition applied to the first substrate, and subjecting the radiation curable composition to photo irradiation which will allow the radiation curable composition to cure to a solid form.

Although other type actinic radiation may be utilized, it is preferable to cure the radiation curable composition using ultraviolet, visible light or black light radiation. In one preferred embodiment, an ultraviolet radiation having a wavelength of about 200 to about 450 nm, preferably about 300 to about 450 nm is used to cure the composition. In another preferred embodiment, the ultraviolet radiation applied to the composition has radiation energy of about 100 mJ/cm² to about 10,000 mJ/cm², preferably about 500 mJ/cm² to about 5,000 mJ/cm². It is preferable for the radiation source to be substantially perpendicular to the substrate during curing.

UV-A-emitting radiation sources (e.g. fluorescent tubes, LED technology or lamps, which are sold for example by Panacol-Elosol GmbH, Steinbach, Germany, under the name UV-H 254, Quick-Start UV 1200, UV-F 450, UV-P 250C, UV-P 280/6 or UV-F 900), high- or medium-pressure mercury vapour lamps, wherein the mercury vapour can be modified by doping with other elements such as gallium or iron, pulsed lamps (known as UV flash lamps) or halogen lamps, for example, are suitable as radiation sources for UV light in the present invention. Further suitable UV emitters or lamps are also can be used in the present invention. The emitters can be installed in a fixed location, such that the item to be irradiated is moved past the radiation source by means of a mechanical device, or the emitters can be mobile and the item to be irradiated does not change its position during the radiation curing.

High- or medium-pressure mercury vapour lamps are preferably used in the method according to the invention, wherein the mercury vapour can be modified by doping with other elements such as gallium or iron.

Generally, the radiation time is preferably short, for example no longer than 5 minutes, preferably no longer than 3 minutes, more preferably no longer than 1 minute.

The radiation curable composition and the cured adhesive/sealant product can be used for bonding articles having substrates made of wood, metal, polymeric plastics, glass and textiles, especially in the manufacture of the liquid crystal displays. In one embodiment, the radiation curable composition and the cured adhesive product according to the present invention are used in the manufacture of the liquid crystal displays by ODF process. A typical ODF process may include the steps of: 1) applying a curable resin composition on a substrate; 2) dropping liquid crystal on a substrate; 3) overlaying the substrates; 4) radiation curing the curable resin composition and obtaining a partially cured product; 5) thermally curing the partially cured product.

The use of the thioxanthone derivative photoinitiator according to the present invention results in an improved curing speed and performance even in shadow/dark region. In addition, the thioxanthone derivative photoinitiator according to the present invention has an excellent compatibility with the resin matrix in the adhesive/sealant composition.

### EXAMPLES

The following examples are intended to assist one skilled in the art to better understand and practice the present invention. The scope of the invention is not limited by the examples but is defined in the appended claims. All parts and percentages are based on weight unless otherwise stated.

### Example 1

### The Synthesis of Photoinitiator (PI) 1 (9-oxo-9H-thioxanthene-2-carboxylic acid dibutylamide)

Carboxylthioxanthone (2.56 g, 10 mmol), 4-dimethylaminopyridine (DMAP) (10 mg), 1-ethyl-(3-dimethylaminopropyl) carbodiimide hydrochloride (EDCl) (1.42 g, 11 mmol) and dibutylamine (1.29 g, 10 mmol) was dissolved in 50 mL dichloromethane (DCM), the solution was stirred at room temperature for 10 hours. The result solution was washed with HCl (100 mL, 1 N) twice, brine (100 mL) once. The pure product having below structure was purified by column chromatograph (ethyl acetate (EA)/petroleum ether (PE)=1:3).

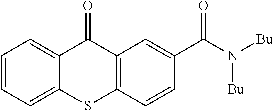

### Example 2

### The Synthesis of PI 2 (2-(morpholine-4-carbonyl)-thioxanthen-9-one)

Carboxylthioxanthone (2.56 g, 10 mmol), DMAP (10 mg), EDCl (1.42 g, 11 mmol) and morpholine (0.87 g, 10 mmol) was dissolved in 50 mL DCM, the solution was stirred at room temperature for 10 hours. The result solution was washed with HCl (100 mL, 1 N) twice, brine (100 mL) once. The pure product having below structure was purified by column chromatograph (EA/PE=1:3).

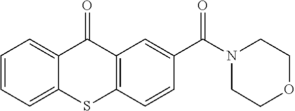 [SMILES: O=C(c1ccc2sc3ccccc3c(=O)c2c1)N1CCOCC1]

### Example 3

### The Synthesis of PI 3 (2-(thiomorpholine-4-carbonyl)-thioxanthen-9-one)

Carboxylthioxanthone (2.56 g, 10 mmol), DMAP (10 mg), EDCl (1.42 g, 11 mmol) and thiomorpholine (1.03 g, 10 mmol) was dissolved in 20 mL DCM, the solution was stirred at room temperature for 10 hours. The result solution was washed with HCl (100 mL, 1 N) twice, brine (100 mL) once. The pure product having below structure was purified by column chromatograph (EA/PE=1:3).

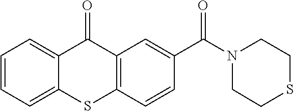 [SMILES: O=C(c1ccc2sc3ccccc3c(=O)c2c1)N1CCSCC1]

### Example 4

### The Synthesis of PI 4 (9-oxo-9H-thioxanthene-2-carboxylic acid 2-diethylamino-ethyl ester)

Carboxylthioxanthone (2.56 g, 10 mmol), DMAP (10 mg), EDCl (1.42 g, 11 mmol) and diethylaminoethanol (1.17 g, 10 mmol) was dissolved in 20 mL DCM, the solution was stirred at room temperature for 10 hours. The result solution was washed with HCl (100 mL, 1 N) twice, brine (100 mL) once. The pure product having below structure was purified by column chromatograph (EA/PE=1:3).

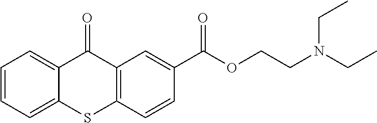 [SMILES: CCN(CC)CCOC(=O)c1ccc2sc3ccccc3c(=O)c2c1]

### Comparative Example

A commercially available photoinitiator 2-isopropylthioxanthone (“ITX”, from Aldrich), was used as Comparative Example.

All examples were tested for the curing properties in various sealant composition systems and the compatibility with resin matrix as below.

### UV Light Curing of Aliphatic Acrylate System

0.1 g photoinitiator of Examples 1 to 4 and Comparative Example (ITX) were each added to 10.0 g butyl acrylate (AR, Sinopharm Chemical Reagent Co., Ltd.). Each sample was mixed with a speed mixer (2350 rpm, 3 min, DAC400FVA, Flack Tech. Inc.). Then UV light curing was applied to each sample by a photo rheometer (320 to 400 nm, 50 mW/cm², mercury lamp (OmniCure 1000), MCR52, Anton Paar), and the time necessary for reaching a certain amount of modulus (G′) of the gradually cured product is shown in Table 1.

TABLE 1
The curing speed in aliphatic acrylate system by UV light curing

| | | | | | Comparative |
|---|---|---|---|---|---|
| | Example 1 | Example 2 | Example 3 | Example 4 | Example |
| G′ | Time | Time | Time | Time | Time |
| kPa | (s) | (s) | (s) | (s) | (s) |
| 30 | 100 | 107 | 103 | 46 | 146 |
| 60 | 107 | 119 | 112 | 53 | 168 |
| 90 | 116 | 128 | 121 | 62 | 188 |
| 120 | 128 | 140 | 131 | 77 | 216 |
| 150 | 159 | 159 | 152 | 113 | 272 |

### UV Light Curing of Aromatic Acrylate System

0.1 g photoinitiator of Examples 1 to 4 and Comparative Example (ITX) were each added to 1.0 g butyl acrylate (AR, Sinopharm Chemical Reagent Co., Ltd.) and 9.0 g bisphenol A glycerolate diacrylate (from Aldrich). Each sample was mixed with a speed mixer (2350 rpm, 3 min, DAC400FVA, Flack Tech. Inc.). Then UV light curing was applied to each sample by a photo rheometer under same conditions as in Test 1. The time necessary for reaching a certain amount of modulus (G′) of the gradually cured product was shown in Table 2.

TABLE 2
The curing speed in aromatic acrylate system by UV light curing

| | | | | | Comparative |
|---|---|---|---|---|---|
| | Example 1 | Example 2 | Example 3 | Example 4 | Example |
| G′ | Time | Time | Time | Time | Time |
| kPa | (s) | (s) | (s) | (s) | (s) |
| 50 | 103 | 124 | 110 | 49 | 154 |
| 150 | 124 | 145 | 128 | 58 | 185 |
| 300 | 142 | 163 | 141 | 67 | 209 |
| 600 | 167 | 182 | 157 | 80 | 236 |
| 900 | 188 | 194 | 167 | 92 | 257 |
| 1200 | 207 | 207 | 177 | 101 | 275 |
| 1500 | 228 | 218 | 186 | 111 | 290 |
| 1800 | 261 | 230 | 195 | 121 | 306 |

### UV Light Curing of Urethane Acrylate System

0.1 g photoinitiator of Examples 1 to 4 and Comparative Example (ITX) were each added to 3.0 g butyl acrylate (AR, Sinopharm Chemical Reagent Co., Ltd.) and 7.0 g EBECRYL 9390 (from Cytec Industries). Each sample was mixed with a speed mixer (2350 rpm, 3 min, DAC400FVA, Flack Tech. Inc.). Then UV light curing was applied to each sample by a photo rheometer under same conditions as in Test 1. The time necessary for reaching a certain amount of modulus (G′) of the gradually cured product was shown in Table 3.

TABLE 3
The curing speed in urethane acrylate system by UV light curing

| | | | | | Comparative |
|---|---|---|---|---|---|
| | Example 1 | Example 2 | Example 3 | Example 4 | Example |
| G′ | Time | Time | Time | Time | Time |
| kPa | (s) | (s) | (s) | (s) | (s) |
| 30 | 52 | 53 | 55 | 50 | 83 |
| 60 | 62 | 64 | 62 | 56 | 110 |
| 90 | 73 | 80 | 71 | 62 | 132 |
| 120 | 89 | 108 | 85 | 73 | 157 |
| 150 | 130 | 168 | 130 | 85 | 194 |

### UV Light Curing of Maleimide Resin System

0.1 g photoinitiator of Examples 1 to 4 and Comparative Example (ITX) were each added to 3.0 g butyl acrylate (AR, Sinopharm Chemical Reagent Co., Ltd.) and 10.0 g N-propylmaleimide (from Aldrich). Each sample was mixed with a speed mixer (2350 rpm, 3 min, DAC400FVA, Flack Tech. Inc.). Then UV light curing was applied to each sample by a photo rheometer under same conditions as in Test 1. The time necessary for reaching a certain amount of modulus (G′) of the gradually cured product was shown in Table 4.

TABLE 4
The curing speed in maleimide system by UV light curing

| | | | | | Comparative |
|---|---|---|---|---|---|
| | Example 1 | Example 2 | Example 3 | Example 4 | Example |
| G′ | Time | Time | Time | Time | Time |
| kPa | (s) | (s) | (s) | (s) | (s) |
| 300 | 106 | 99 | 44 | 32 | 203 |
| 500 | 113 | 103 | 47 | 35 | 227 |
| 1000 | 125 | 115 | 53 | 38 | 264 |
| 1500 | 137 | 125 | 59 | 41 | 294 |
| 2000 | 151 | 137 | 67 | 44 | 320 |

### Visible Light Curing of Maleimide Type Resin System

0.1 g photoinitiator of Examples 1 to 4 and Comparative Example (ITX) were each added to 3.0 g butyl acrylate (AR, Sinopharm Chemical Reagent Co., Ltd.) and 10.0 g camphorquinone (from Aldrich). Each sample was mixed with a speed mixer (2350 rpm, 3 min, DAC400FVA, Flack Tech. Inc.). Then UV light curing was applied to each sample by a photo rheometer (405 nm, 50 mW/cm², LED lamp (OmniCure 1000), MCR52, Anton. Paar). The time necessary for reaching a certain amount of modulus (G′) of the gradually cured product was shown in Table 5.

TABLE 5
The curing speed in maleimide system by visible light curing

| | | | | | Comparative |
|---|---|---|---|---|---|
| | Example 1 | Example 2 | Example 3 | Example 4 | Example |
| G′ | Time | Time | Time | Time | Time |
| kPa | (s) | (s) | (s) | (s) | (s) |
| 100 | 128 | 98 | 98 | 36 | 277 |
| 150 | 134 | 101 | 101 | / | 289 |
| 300 | 145 | 110 | 107 | 39 | 317 |
| 500 | 157 | 116 | 115 | 41 | 343 |
| 1000 | 183 | 134 | 130 | 50 | 396 |
| 1500 | 215 | 155 | 146 | 64 | 441 |
| 2000 | 263 | 182 | 167 | 97 | 485 |

Shadow Cure Test with UV Light

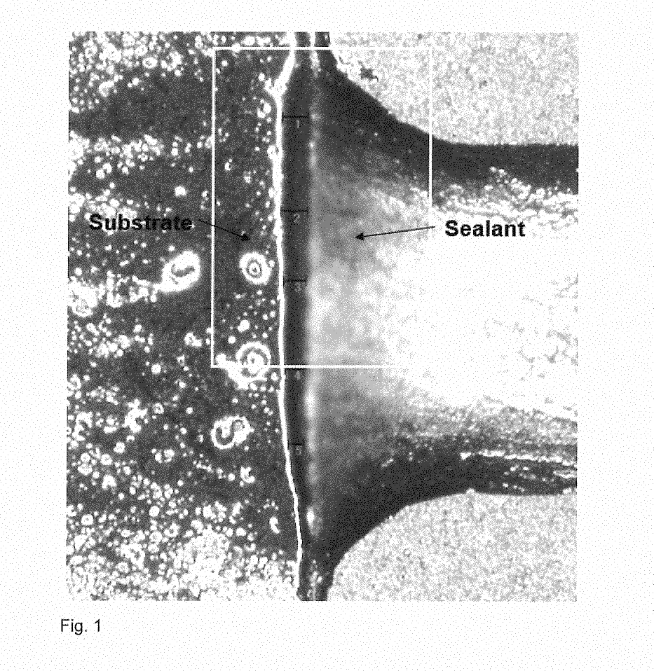

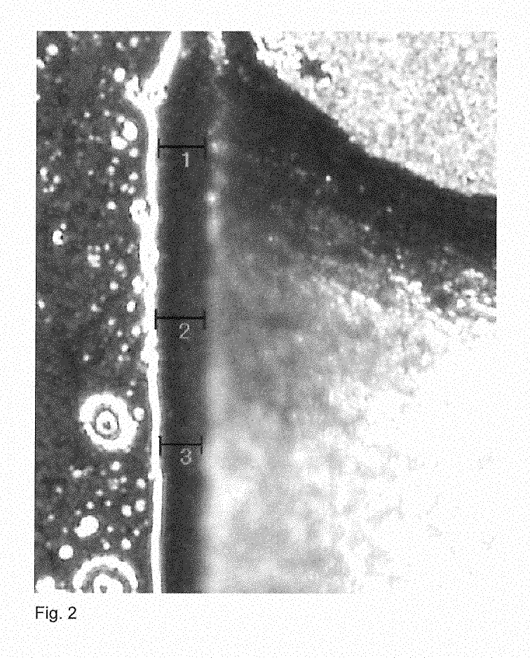

0.1 g photoinitiator of Examples 1 to 4 and Comparative Example (ITX) were each added to 10.0 g N-propylmaleimide (from Aldrich). Each sample was mixed with a speed mixer (2350 rpm, 3 min, DAC400FVA, Flack Tech. Inc.). Then UV light curing was applied to each sample by a photo rheometer (365 nm, 3000 mJ/cm², mercury lamp, EW50AAG, Fuji Electric FA). The curing depth was measured by a microscope (MX61, Olympus). As shown in FIGS. 1 and 2, the average of five curing depths, defining the widths of the cured product in no light exposure area, were calculated and recorded in Table 6.

TABLE 6
The curing depth in shadow cure by UV light

| | | | | | Comparative |
|---|---|---|---|---|---|
| | Example 1 | Example 2 | Example 3 | Example 4 | Example |
| Curing | 30 | 32 | 38 | 34 | 13 |
| depth | | | | | |
| (μm) | | | | | |

Shadow Cure Test with Visible Light

0.1 g photoinitiator of Examples 1 to 4 and Comparative Example (ITX) were each added to 10.0 g N-propylmaleimide (from Aldrich). Each sample was mixed with a speed mixer (2350 rpm, 3 min, DAC400FVA, Flack Tech. Inc.). Then UV light curing was applied to each sample by a photo rheometer (405 nm, 20 J/cm², LED lamp LED flood, Loctite). The curing depth was measured by a microscope (MX61, Olympus). As shown in FIGS. 1 and 2, the average of five curing depths were calculated and recorded in Table 7.

TABLE 7
The curing depth in shadow cure by visible light

| | | | | | Comparative |
|---|---|---|---|---|---|
| | Example 1 | Example 2 | Example 3 | Example 4 | Example |
| Curing | 230 | 165 | 125 | 335 | 100 |
| depth | | | | | |
| (μm) | | | | | |

### Miscibility in the Radically Curable Sealant Composition

0.1 g photoinitiator of Examples 1 to 4 and Comparative Example (ITX) were each added to 2.0 g N-propylmaleimide and 5.0 g EBECRYL 9390, and then 3.0 g Hardener 5923 (from Ashahi Kasei) was added in the mixture. Each sample was mixed with a speed mixer (2350 rpm, DAC400FVA, Flack Tech. Inc.) under room temperature. The sample was checked in every 30 seconds if the photoinitiator was dissolved. When a complete dissolution was observed, the time after the start of mixing was recorded as the dispersion time in Table 8.

TABLE 8
The results of dispersion time

| | | | | | Comparative |
|---|---|---|---|---|---|
| | Example 1 | Example 2 | Example 3 | Example 4 | Example |
| Dispersion | 30 | 120 | 120 | 30 | 300 |
| time(s) | | | | | |

As demonstrated in Tables 1 to 5, the sealant composition containing the photoinitiators according to the present invention exhibited a much faster curing speed than the comparative example in various resin matrix systems including aliphatic (metha)acrylate, aromatic (metha)acrylate, urethane(metha)acrylate, maleimide resin by UV light curing and visible light curing.

In addition, as shown in Tables 6 and 7, the sealant composition containing the photoinitiators according to the present invention achieved an improved curing depth than that of the comparative example in shadow cure conditions with UV light curing and visible light curing. Such excellent behavior in shadow cure achieved by the present invention allowed the sealant compositions to avoid the liquid crystal penetration and contamination during the liquid crystal assembly process, such as one-drop-fill process.

Further, as shown in Table 8, the photoinitiators according to the present invention possessed an excellent miscibility with other components such as resin matrix, hardener in the sealant composition even under room temperature, and thus provided the ease for industrial use as additional heating step for obtaining a homogenous composition is not necessary, which facilitates in the large scale production of liquid crystal devices.

What is claimed is:

1. A thioxanthone derivative photoinitiator, represented by general formula (1): 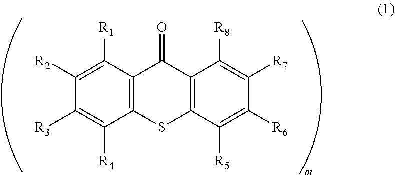
wherein m is 1 or 2;
R₁ to R₈ each independently represents hydrogen, halogen, an optionally substituted alkyl group, an optionally substituted alkenyl group, an optionally substituted alkoxy group, an optionally substituted aralkyl group, an optionally substituted aryl group, or an optionally substituted heteroaryl group, and at least one of R₁ to R₈ represents a moiety of formula (2), 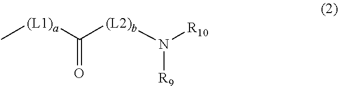
wherein L1 and L2 each independently represents an optionally substituted alkylene group, or an optionally substituted —Y-alkylene group, wherein the alkylene chain is optionally interrupted by —N(R₁₁)—, —O—, —S—, —S(O)—, —S(O)₂—, —C(O)—, —C(O)O—, —C(O)N(R₁₁)—, —S(O)₂N(R₁₁)—, —OC(O)N(R₁₁)—, —N(R₁₁)C(O)—, —N(R₁₁)SO₂—, —N(R₁₁)C(O)O—, —N(R₁₁)C(O)N(R₁₁)—, —N(R₁₁)S(O)₂N(R₁₁)—, —OC(O)—, or —C(O)N(R₁₁)—O—, in which R₁₁ is hydrogen or an optionally substituted C₁₋₁₂ aliphatic group, and Y represents an optionally substituted heteroatom;
a, b is 0, 1 or 2; and
R₉ and R₁₀ each independently represents an optionally substituted alkyl group, an optionally substituted alkoxyl group, an optionally substituted alkenyl group, an optionally substituted alkynyl group, an optionally substituted aralkyl group, an optionally substituted aryl group, or an optionally substituted heteroaryl group, or R₉ and R₁₀ taken together may form a 5- to 8-membered carbocyclic or hetero atom-containing ring.

2. The thioxanthone derivative photoinitiator according to claim 1, wherein any of R₅ to R₈ represents a moiety of formula (2), and the others of R₁ to R₈ each independently represents hydrogen, halogen, an alkyl group, an alkoxy group, an aralkyl group, an aryl group, or a heteroaryl group.

3. The thioxanthone derivative photoinitiator according to claim 1, wherein R₉ and R₁₀ each independently represents an optionally substituted alkyl group, an optionally substituted alkenyl group, an optionally substituted alkynyl group, an optionally substituted aralkyl group, an optionally substituted aryl group, or an optionally substituted heteroaryl group.

4. The thioxanthone derivative photoinitiator according to claim 1, wherein R₉ and R₁₀ taken together may form a 5- to 8-membered, preferably 6-membered carbocyclic or hetero atom-containing ring.

5. The thioxanthone derivative photoinitiator according to claim 1, represented by general formula (3), 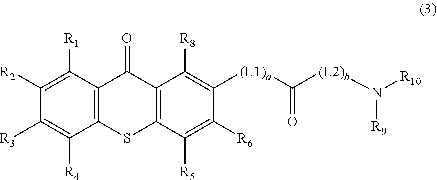
wherein,
R₁ to R₆ and R₈ each independently represents hydrogen, halogen, an optionally substituted alkyl group, an optionally substituted alkenyl group, an optionally substituted alkoxy group, an optionally substituted aralkyl group, an optionally substituted aryl group, or an optionally substituted heteroaryl group;
L1 and L2 each independently represents an optionally substituted alkylene group, or an optionally substituted -Y-alkylene group, wherein the alkylene chain is optionally interrupted by —N(R₁₁)—, —O—, —S—, —S(O)—, —S(O)₂—, —C(O)—, —C(O)O—, —C(O)N(R₁₁)—, —S(O)₂N(R₁₁)—, —OC(O)N(R₁₁)—, —N(R₁₁)C(O)—, —N(R₁₁)SO₂—, —N(R₁₁)C(O)O—, —N(R₁₁)C(O)N(R₁₁)—, —N(R₁₁)S(O)₂N(R₁₁)—, —OC(O)—, or —C(O)N(R₁₁)—O—, in which R₁₁ is hydrogen or an optionally substituted C₁₋₁₂ aliphatic group, and Y represents an optionally substituted heteroatom;
a, b is 0, 1 or 2;
R₉ and R₁₀ each independently represents an optionally substituted alkyl group, an optionally substituted alkenyl group, an optionally substituted alkynyl group, an optionally substituted aralkyl group, an optionally substituted aryl group, or an optionally substituted heteroaryl group.

6. The thioxanthone derivative photoinitiator according to claim 1, represented by general formula (4), 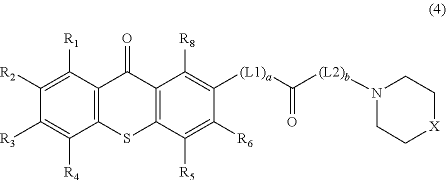
wherein,
R₁ to R₆ and R₈ each independently represents hydrogen, halogen, an optionally substituted alkyl group, an optionally substituted alkenyl group, an optionally substituted alkoxy group, an optionally substituted aralkyl group, an optionally substituted aryl group, or an optionally substituted heteroaryl group;
L1 and L2 each independently represents an optionally substituted alkylene group, or an optionally substituted -Y-alkylene group, wherein the alkylene chain is optionally interrupted by —N(R₁₁)—, —O—, —S—, —S(O)—, —S(O)₂—, —C(O)—, —C(O)O—, —C(O)N(R₁₁)—, —S(O)₂N(R₁₁)—, —OC(O)N(R₁₁)—, —N(R₁₁)C(O)—, —N(R₁₁)SO₂—, —N(R₁₁)C(O)O—, —N(R₁₁)C(O)N(R₁₁)—, —N(R₁₁)S(O)₂N(R₁₁)—, —OC(O)—, or —C(O)N(R₁₁)—O—, in which R₁₁ is hydrogen or an optionally substituted C₁₋₁₂ aliphatic group, and Y represents an optionally substituted heteroatom;
a, b is 0, 1 or 2; and
X represents hydrogen, or an optionally substituted heteroatom.

7. The thioxanthone derivative photoinitiator according to claim 1, wherein a is 0, and b is 0 or 1.

8. The thioxanthone derivative photoinitiator according to claim 1, wherein when b is not 0, L2 is an optionally substituted C₁₋₆ alkoxylene group.

9. The thioxanthone derivative photoinitiator according to claim 1, wherein R₉ and R₁₀ each represents a C₁₋₁₂ alkyl group or C₁₋₁₂ alkoxyl group.

10. The thioxanthone derivative photoinitiator according to claim 1, wherein X is optionally substituted, preferably alkyl substituted nitrogen, oxygen or sulfur.

11. The thioxanthone derivative photoinitiator according to claim 1, selected from the group consisting of:

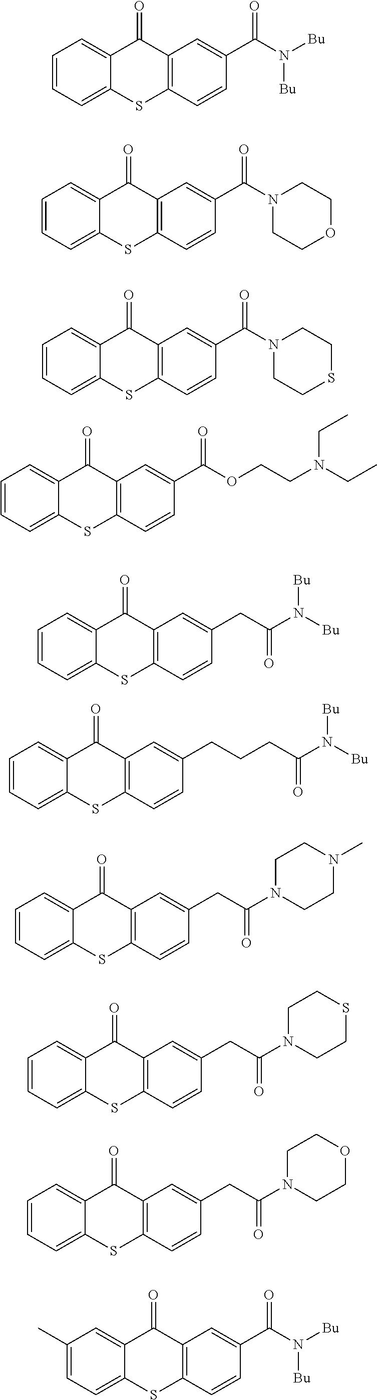

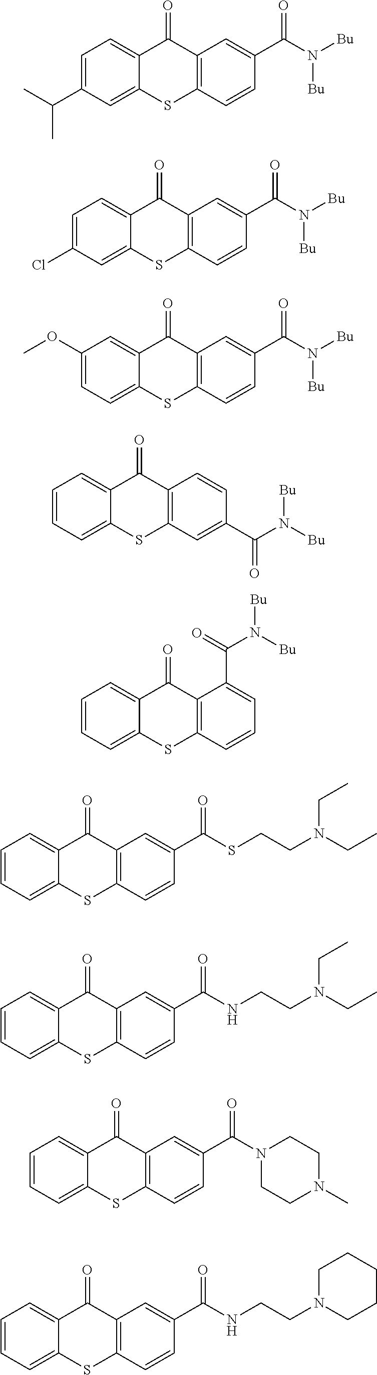

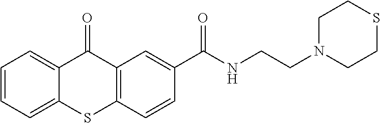 [SMILES: O=C(NCCN1CCSCC1)c1ccc2sc3ccccc3c(=O)c2c1]

12. A radiation curable composition comprising the thioxanthone derivative photoinitiator according to claim 1.

13. A cured adhesive or sealant product obtained from the radiation curable composition according to claim 12.

14. A method of bonding materials together which comprises applying the radiation curable composition according to claim 12 in a liquid form to a first substrate, bringing a second substrate in contact with the radiation curable composition applied to the first substrate, and subjecting the radiation curable composition to photo irradiation which will allow the radiation curable composition to cure to a solid form.
